(19) United States
(12) Patent Application Publication
(10) Pub. No.: US 2017/0196893 A1
(43) Pub. Date: Jul. 13, 2017

(54) TREATMENT OF INFLAMMATORY DEMYELINATING DISEASE WITH AGONISTS OF FARNESOID X RECEPTOR

(71) Applicant: The Board of Trustees of the Leland Stanford Junior University, Stanford, CA (US)

(72) Inventors: Lawrence Steinman, Stanford, CA (US); Peggy P. Ho, Cupertino, CA (US)

(21) Appl. No.: 15/400,903

(22) Filed: Jan. 6, 2017

Related U.S. Application Data
(60) Provisional application No. 62/276,034, filed on Jan. 7, 2016.

(51) Int. Cl. A61K 31/575; A61K 45/06; A61K 9/00

(52) U.S. Cl. CPC A61K 31/575; A61K 9/0053; A61K 45/06

(57) ABSTRACT

Provided herein are methods and compositions for treating demyelinating inflammatory diseases by administering to the subject an effective dose on an FXR agonist.

### CROSS REFERENCE

This application claims benefit of U.S. Provisional Patent Application No. 62/276,034, filed Jan. 7, 2016, which application is incorporated herein by reference in its entirety.

### BACKGROUND

Bile acid synthesis plays a major role in cholesterol homeostasis in the liver and small intestine. Within the liver, bile acids are synthesized from cholesterol and serve as the final end products of cholesterol catabolism. Bile acids are also essential for the adsorption and solubilization of dietary cholesterol and fat-soluble vitamins. When high levels of bile acids accumulate in the body, the nuclear bile acid receptor, farnesoid X receptor (FXR), is activated by its natural bile acid ligands that include chenodeoxycholic acid (CDCA), deoxycholic (DCA), and lithocholic (LCA). This negatively regulates the transcription of the rate-limiting enzyme, cholesterol 7a-hydroxylase (CYP7A1) to inhibit further bile acid synthesis and uptake and to increase the export of bile acids out of the cells. CDCA is the highest affinity natural ligand for FXR.

FXR is mainly expressed in liver, intestine, kidneys, and adrenal gland, with less expression in adipose tissue and heart. FXR was originally identified as a receptor for the cholesterol precursor farnesol, but is now recognized as the primary receptor for bile acid. The bile acid-FXR interaction plays an integral role in regulating hepatic inflammation and regeneration, and FXR activation has been shown to attenuate liver injury in a rodent model of autoimmune hepatitis. In the intestinal tract, bile acids and FXR prevent bacterial overgrowth and regulate the extent of inflammatory responses and pathology from a compromised barrier. Furthermore, FXR activation results in preservation of the intestinal barrier in a rodent model of inflammatory bowel disease. Activation of FXR has been reported to inhibit vascular smooth muscle cell (VSMC) inflammation by down-regulating the proinflammatory enzymes inducible nitric oxide synthase and cyclooxygenase-2 expression as well as cell migration into VSMCs. This suggests that FXR may be a potential target for the progressive inflammatory disease, atherosclerosis. Recently, twelve nuclear receptors including FXR, were shown to be expressed on highly purified CD4+, CD8+, CD19+ and CD14+ cells by quantitative realtime PCR, suggesting that FXR may be co-regulated in human immune cells.

Obeticholic acid (6-ECDCA), an orally active synthetic FXR agonist, has ^{˜}100-fold greater FXR agonistic activity than CDCA and does not activate other nuclear receptors. In animal models, 6-EDCA has been shown to treat cholestatic liver disease and decrease insulin resistance and hepatic steatosis. 6-ECDCA is currently in phase I clinical trials for the treatment of alcoholic hepatitis, phase II clinical trials for nonalcoholic steatohepatitis (NASH) and type 2 diabetes mellitus, and phase III clinical trials of primary biliary cirrhosis (PBC).

### SUMMARY

Provided herein are therapeutic methods for the treatment of neurological inflammatory diseases, such as, for example, demyelinating autoimmune diseases, such as multiple sclerosis and neuromyelitis optica, etc. In the methods of the invention, an effective dose of one or a cocktail of FXR agonist(s) is administered to a subject suffering from a demyelinating autoimmune disease. In some embodiments the agonist is a synthetic agonist, including without limitation obeticholic acid. In other embodiments the FXR agonist is a natural agonist, including without limitation one or more of chenodeoxycholic acid (CDCA), deoxycholic (DCA), and lithocholic (LCA). In some embodiments, administration is oral. In various aspects and embodiments, the methods may include administering to a patient an effective dose of an agonist of farnesoid X receptor (FXR). In some embodiments the FXR agonist is combined with a statin in a dose effective to control serum cholesterol levels.

In one embodiment, provided is a package (for example a box, a bottle or a bottle and box) that includes an agonist of FXR and a package insert or label that indicates that the FXR agonist is to be administered to a patient for the treatment of a demyelinating autoimmune disease (such as multiple sclerosis). In some embodiments the agonist is a synthetic agonist, including without limitation obeticholic acid. In other embodiments the FXR agonist is a natural agonist, including without limitation one or more of chenodeoxycholic acid (CDCA), deoxycholic (DCA), and lithocholic (LCA). In some embodiments, administration is oral. In some embodiments the FXR agonist is combined with a statin in a dose effective to control serum cholesterol levels.

In one aspect, provided is a composition for oral administration (e.g., a pill, capsule, tablet, syrup, emulsion, liquid, elixir and the like) of an FXR agonist for the treatment of demyelinating autoimmune disease.

In one embodiment, provided is a method of treating a demyelinating autoimmune disease (such as multiple sclerosis) that includes administering to a patient an effective dose of an agonist of farnesoid X receptor (FXR) alone or in combination with a statin, in combination with one or more therapeutic compounds, including without limitation a cytokine; an antibody, e.g. tysabri; fingolimod (Gilenya); copaxone, etc. The effective dose of each drug in a combination therapy may be lower than the effective dose of the same drug in a monotherapy. In some embodiments the combined therapies are administered concurrently. In some embodiments the two therapies are phased, for example where one compound is initially provided as a single agent, e.g. as maintenance, and where the second compound is administered during a relapse, for example at or following the initiation of a relapse, at the peak of relapse, etc. Alternatively the FXR agonist is initially provided as a single agent, e.g. as maintenance, and the additional agent is administered during a relapse, for example at or following the initiation of a relapse, at the peak of relapse, etc. In certain of such embodiments, a package is provided comprising includes an FXR agonist, and one or more second therapeutic compounds, and a package insert or label that indicates that the FXR agonist is to be administered in combination with the second compound to a patient for the treatment of a demyelinating autoimmune disease (such as multiple sclerosis).

In some embodiments of the invention, the patient is analyzed for responsiveness to therapy, where the selection of therapeutic agents is based on such analysis. The efficacy of immunomodulatory treatments of inflammatory demyelinating diseases of the central nervous system, e.g. multiple sclerosis, neuromyelitis optica, EAE, etc., depends on whether a patient has a predominantly TH1-type disease subtype, or a predominantly TH17-type disease subtype. Patients can be classified into subtypes by determining the levels of markers, including IL-17; endogenous β-interferon, IL-23, PDGFBB, sFAS ligand, M-CSF, MIP1α, TNF-β, IFNα, IL-1RA, MCP-1, IL-2, IL-6, IL-8, FGFβ, IL-7, TGF-β, IFNβ, IL-13, IL-17F, EOTAXIN, IL-1a, MCP-3, LIF, NGF, RANTES, IL-5, MIP1b, IL-12p70, and HGF, etc. Cytokines such as β-interferon may be administered to individuals having a predominantly TH1-type disease subtype in combination with an FXR agonist.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. FXR Knockout mice have more severe EAE. (A) Serum lipid panel analysis confirms that FXR-KO mice have elevated serum cholesterol and triglyceride levels after fasting overnight. (B) Clinical EAE scores of wildtype (WT, n=8) versus FXR-knockout (FXR-KO, n=8) mice immunized with MOG35-55 in CFA with pertussis toxin. Each point represents mean+/− SEM, * represents p<0.05 by Mann-Whitney U test. (C) Seventy-two hour proliferation assay of harvested splenocytes with increasing concentrations of MOG35-55. ³H-Thymidine incorporation is measured in triplicate wells. Values are the mean+SEM of triplicates, * represents p<0.05 by Student t-test. These experiments were repeated once with similar results.

FIG. 2. Oral administration of Obeticholic Acid is most effective in treating EAE than intraperitoneal injections. (A) Clinical EAE scores of mice administered 6-ECDCA or vehicle. Mice with established EAE were randomized into three groups (n=9) and given daily doses of vehicle or 5 mg/kg of 6-ECDCA either intraperitoneally (i.p.) or orally (oral). Each point represents mean+/−SEM, * represents p<0.05 by Mann-Whitney U test. (B) Seventy-two hour proliferation assay of harvested splenocytes with increasing concentrations of MOG35-55. 3H-Thymidine incorporation is measured in triplicate wells. Values are the mean+SEM of triplicates, * represents p<0.05 by Student's t-test. (C) Cytokine production by harvested splenocytes with increasing concentrations of MOG35-55. Values are the mean+SEM of triplicates, * represents p<0.05 by Student's t-test.

FIG. 3. Obeticholic Acid is more effective than Chenodeoxycholic acid in treating EAE. (A) Chemical structures of CDCA and 6-ECDCA. (B) Clinical EAE scores of mice administered 6-ECDCA, CDCA, or vehicle. Mice with established EAE were randomized into three groups (n=9) and given daily oral doses of vehicle or 5 mg/kg of 6-ECDCA or CDCA. Each point represents mean+/−SEM, * represents p<0.05 by Mann-Whitney U test. (C) Seventy-two hour proliferation assay of harvested splenocytes with increasing concentrations of MOG35-55. 3H-Thymidine incorporation is measured in triplicate wells. Values are the mean+SEM of triplicates, * represents p<0.05 by Student's t-test. (D) Cytokine production by harvested splenocytes with increasing concentrations of MOG35-55. Values are the mean+SEM of triplicates, * represents p<0.05 by Student's t-test. These experiments were repeated twice with similar results.

FIG. 4. FXR agonists alter the lymphocyte activation profile in EAE and prevent EAE disease transfer. (A) Serum lipid panel analysis comparing naïve (n=5), vehicle (n=5), CDCA (n=4) and 6-ECDCA (n=5) treated MOG35-55-immunized mice given eleven doses daily and fasted overnight. (B) Seventy two hour proliferation assay of harvested splenocytes with increasing concentrations of MOG35-55. 3H-Thymidine incorporation is measured in triplicate wells. Values are the mean+SEM of triplicates, * represents p<0.05 by Student's t-test. (C) Graphs represent summarized FACS analysis of harvested splenocytes profiling CD4+ T cells, CD8+ T cells, CD19+ B cells and their expression of PD1, PD-L1, and BTLA, and VLA-4 (CD29⁺CD49d⁺) expression on T and B cells. (D) Clinical EAE scores of naïve recipient mice (each group, n=15) administered 3×10⁷ donor spleen and lymph node cells from mice treated with either 6-ECDCA, CDCA, or vehicle for eleven days before in vitro restimulation with 20 μg/ml MOG³⁵⁻⁵⁵ and 10 ng/ml rIL-12 for 72 hours. Each point represents mean+/−SEM, * represents p<0.05 by Mann-Whitney U test. (E) Survival curve of EAE mice shown in panel D.

### DETAILED DESCRIPTION OF THE EMBODIMENTS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The present inventions have been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

Improvement in the use of disease-modifying therapies in autoimmune conditions is of great clinical interest. In certain aspects and embodiments the present methods and compositions address this need.

The subject methods may be used for prophylactic or therapeutic purposes. As used herein, the term “treating” is used to refer to both prevention of relapses, and treatment of pre-existing conditions. For example, the prevention of autoimmune disease may be accomplished by administration of the agent prior to development of a relapse. The treatment of ongoing disease, where the treatment stabilizes or improves the clinical symptoms of the patient, is of particular interest.

“Diagnosis” as used herein generally includes determination of a subject's susceptibility to a disease or disorder, determination as to whether a subject is presently affected by a disease or disorder, prognosis of a subject affected by a disease or disorder (e.g., identification of disease states, stages of MS, or responsiveness of MS to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood, cerebral spinal fluid, and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, for example humans, non-human primate, mouse, rat, guinea pig, rabbit, etc.

“Inhibiting” the onset of a disorder shall mean either lessening the likelihood of the disorders onset, or preventing the onset of the disorder entirely. Reducing the severity of a relapse shall mean that the clinical indicia associated with a relapse are less severe in the presence of the therapy than in an untreated disease. As used herein, onset may refer to a relapse in a patient that has ongoing relapsing remitting disease. The methods of the invention are specifically applied to patients that have been diagnosed with an autoimmune disease. Treatment is aimed at the treatment or reducing severity of relapses, which are an exacerbation of a pre-existing condition.

“Inhibiting” the expression of a gene in a cell shall mean either lessening the degree to which the gene is expressed, or preventing such expression entirely.

Inflammatory demyelinating disease. The term “inflammatory” response is the development of a humoral (antibody mediated) and/or a cellular (mediated by antigen-specific T cells or their secretion products) response. Inflammatory demyelinating diseases of the central nervous system are of particular interest and include, without limitation, multiple sclerosis (MS), neuromyelitis optica (NO), and experimental acquired encephalitis (EAE). Demyelinating inflammatory diseases of the peripheral nervous system include Guillain-Barre syndrome (GBS) with its subtypes acute inflammatory demyelinating polyradiculoneuropathy, acute motor axonal neuropathy, acute motor and sensory axonal neuropathy, Miller Fisher syndrome, and acute pandysautonomia; chronic inflammatory demyelinating polyneuropathy (CIDP) with its subtypes classical CIDP, CIDP with diabetes, CIDP/monoclonal gammopathy of undetermined significance (MGUS), sensory CIDP, multifocal motor neuropathy (MMN), multifocal acquired demyelinating sensory and motor neuropathy or Lewis-Sumner syndrome, multifocal acquired sensory and motor neuropathy, and distal acquired demyelinating sensory neuropathy.

Multiple sclerosis is characterized by various symptoms and signs of CNS dysfunction, with remissions and recurring exacerbations. Classifications of interest for analysis by the methods of the invention include relapsing remitting MS (RRMS), primary progressive MS (PPMS) and secondary progressive MS (SPMS). The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat can accentuate symptoms and signs.

The course is highly varied, unpredictable, and, in most patients, remittent. At first, months or years of remission can separate episodes, especially when the disease begins with retrobulbar optic neuritis. However, some patients have frequent attacks and are rapidly incapacitated; for a few the course can be rapidly progressive (primary progressive MS, PPMS), or secondary progressive multiple sclerosis (SPMS). Relapsing remitting MS (RR MS) is characterized clinically by relapses and remissions that occur over months to years, with partial or full recovery of neurological deficits between attacks. Such patients manifest approximately 1 attack, or relapse, per year. Over 10 to 20 years, approximately 50% of RR MS patients develop secondary progressive MS (SP MS) which is characterized by incomplete recovery between attacks and accumulation of neurologic deficits resulting in increasing disability.

Diagnosis is usually indirect, by deduction from clinical, radiographic (brain plaques on magnetic resonance [MR] scan), and to a lesser extent laboratory (oligoclonal bands on CSF analysis) features. Typical cases can usually be diagnosed confidently on clinical grounds. The diagnosis can be suspected after a first attack. Later, a history of remissions and exacerbations and clinical evidence of CNS lesions disseminated in more than one area are highly suggestive.

MRI, the most sensitive diagnostic imaging technique, can show plaques. It can also detect treatable nondemyelinating lesions at the junction of the spinal cord and medulla (eg, subarachnoid cyst, foramen magnum tumors) that occasionally cause a variable and fluctuating spectrum of motor and sensory symptoms, mimicking MS. Gadolinium-contrast enhancement can distinguish areas of active inflammation from older brain plaques. MS lesions can also be visible on contrast-enhanced CT scans; sensitivity can be increased by giving twice the iodine dose and delaying scanning (double-dose delayed CT scan).

Neuromyelitis optica (NMO), or Devic's disease, is an autoimmune, inflammatory disorder of the optic nerves and spinal cord. Although inflammation can affect the brain, the disorder is distinct from multiple sclerosis, having a different pattern of response to therapy, possibly a different pattern of autoantigens and involvement of different lymphocyte subsets.

The main symptoms of Devic's disease are loss of vision and spinal cord function. As for other etiologies of optic neuritis, the visual impairment usually manifests as decreased visual acuity, although visual field defects, or loss of color vision can occur in isolation or prior to formal loss of acuity. Spinal cord dysfunction can lead to muscle weakness, reduced sensation, or loss of bladder and bowel control. The damage in the spinal cord can range from inflammatory demyelination to necrotic damage of the white and grey matter. The inflammatory lesions in Devic's disease have been classified as type II lesions (complement mediated demyelinization), but they differ from MS pattern II lesions in their prominent perivascular distribution. Therefore, the pattern of inflammation is often quite distinct from that seen in MS.

Attacks are conventionally treated with short courses of high dosage intravenous corticosteroids such as methylprednisolone IV. When attacks progress or do not respond to corticosteroid treatment, plasmapheresis can be used. Commonly used immunosuppressant treatments include azathioprine (Imuran) plus prednisone, mycophenolate mofetil plus prednisone, Rituximab, Mitoxantrone, intravenous immunoglobulin (IVIG), and cyclophosphamide.

The disease can be monophasic, i.e. a single episode with permanent remission. However, at least 85% of patients have a relapsing form of the disease with repeated attacks of transverse myelitis and/or optic neuritis. In patients with the monophasic form the transverse myelitis and optic neuritis occur simultaneously or within days of each other. Patients with the relapsing form are more likely to have weeks or months between the initial attacks and to have better motor recovery after the initial transverse myelitis event. Relapses usually occur early with about 55% of patients having a relapse in the first year and 90% in the first 5 years. Unlike MS, Devic's disease rarely has a secondary progressive phase in which patients have increasing neurologic decline between attacks without remission. Instead, disabilities arise from the acute attacks.

Farnesoid x receptor (FXR) is a member of the nuclear receptor superfamily of ligand-regulated transcription factors. Bile acids, the end product of cholesterol catabolism, are the physiological ligands for FXR and act to stimulate FXR-dependent gene expression. FXR thus functions as a bile acid sensor that impacts the regulation of genes involved in cholesterol, triglyceride (TG), and bile acid production to maintain lipid homeostasis.

Studies in both humans and animals show that modulation of the bile acid pool through binding resins or bile acid supplementation can have profound effects on plasma TG and cholesterol levels. Treatment with the bile acid chenodeoxycholic acid (CDCA) to patients for the dissolution of gallstones resulted in a concomitant decrease in circulating TG levels and VLDL production. An FXR agonist may also elevate serum cholesterol levels.

Agonists of FXR are known in the art, and include synthetic and naturally occurring molecules. Examples of natural agonists include, for example, chenodeoxycholic acid (CDCA), deoxycholic (DCA), and lithocholic (LCA).

Examples of synthetic FXR agonists include, for example, WAY-362450 (FXR-450/XL335); 6alpha-ethyl-chenodeoxycholic acid (6-ECDCA); AGN-34; UDCA (ursofalk, ursodiol); 3-[2-[2-Chloro-4-[[3-(2,6-dichlorophenyl)-5-(1-methylethyl)-4-isoxazolyl] methoxy]phenyl]ethenyl]benzoic acid (GW4064); fexaramine, see for example Pellicciari et al. (2002) J. Med. Chem. 45(17):3569-72; and Bassa et al. (2009) Bioorganic & Medicinal Chemistry Letters 19(11):2969-2973, each herein specifically incorporated by reference.

Statins are inhibitors of HMG-CoA reductase enzyme. These agents are described in detail, for example, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171; fluvastatin and related compounds as disclosed in U.S. Pat. No. 5,354,772; atorvastatin and related compounds as disclosed in U.S. Pat. Nos. 4,681,893, 5,273,995 and 5,969,156; and cerivastatin and related compounds as disclosed in U.S. Pat. Nos. 5,006,530 and 5,177,080. Additional compounds are disclosed in U.S. Pat. Nos. 5,208,258, 5,130,306, 5,116,870, 5,049,696, RE 36,481, and RE 36,520.

An effective dose of a statin is the dose that, when administered for a suitable period of time, usually at least about one week, and may be about two weeks, or more, up to a period of about 4 weeks, will evidence a reduction in the severity of the disease and/or control serum cholesterol levels. It will be understood by those of skill in the art that an initial dose may be administered for such periods of time, followed by maintenance doses, which, in some cases, will be at a reduced dosage.

The formulation and administration of statins is well known, and will generally follow conventional usage. The dosage required to treat autoimmune disease may be the same or may vary from the levels used for management of cholesterol in the absence of FXR agonist treatment.

Statins can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. The formulation is optionally combined in a unit dose with an FXR agonist.

Interferon beta is a drug in the interferon family used to treat multiple sclerosis (MS). IFN-β1a is produced by mammalian cells while Interferon beta-1b is produced in modified E. coli. Interferons have been shown to have about a 18-38% reduction in the rate of MS relapses, and to slow the progression of disability in MS patients. Commercially available products include Avonex (Biogen Idec); Rebif (EMD Serono); and CinnoVex (CinnaGen). Closely related is Interferon beta-1b, which is marketed in the US as Betaseron, or Extavia.

Various formulations and dosages are conventionally utilized in the treatment of MS patients with IFN-β, which doses may be utilized in the combination treatments of the present invention, or may be utilized at a lower dose, e.g. 90% of the conventional dose, 80% of the conventional dose, 70% of the conventional dose, 60% of the conventional dose, 50% of the conventional dose, or less.

Avonex is sold in two formulations, a lyophilized powder requiring reconstitution and a pre-mixed liquid syringe kit; it is usually administered once per week via intramuscular injection at a dose of 30 μg. Rebif is administered via subcutaneous injection three times per week at a dose of 22 μg or 44 μg. Interferon beta-1b is usually administered at 250 μg on alternate days.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

A “subject” or “patient” in the context of the present teachings is generally a mammal. Mammals other than humans can be advantageously used as subjects that represent animal models of inflammation. A subject can be male or female.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis to, e.g., determine whether a subject is a responder or a non-responder to a therapy (e.g., an IFN treatment as described herein).

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

### Methods

The present disclosure provides methods for treating neurological inflammatory diseases, which may be a demyelinating autoimmune disease, such as multiple sclerosis. The methods comprise administering to the subject an effective amount of an agent that is an agonist of FXR. The FXR agonist can be a naturally occurring or synthetic agonist. In certain embodiments In some embodiments the agonist is a synthetic agonist, including without limitation obeticholic acid. In some embodiments administration is oral.

In certain embodiments the FXR agonist is combined with a therapeutic dose of a statin. The active agents may be administered in separate formulations, or may be combined, e.g. in a unit dose. The formulation may be for oral administration.

Optionally the FXR agonist is combined as a single agent or with a statin in a combination with a second compound such as a cytokine; an antibody, e.g. tysabri; fingolimod (Gilenya); copaxone, etc. In some embodiments the cytokine is IFN-β. In some embodiments the combined therapies are administered concurrently, where the administered dose of any one of the compounds may be a conventional dose, or less than a conventional dose. In some embodiments the two therapies are phased, for example where one compound is initially provided as a single agent, e.g. as maintenance, and where the second compound is administered during a relapse, for example at or following the initiation of a relapse, at the peak of relapse, etc.

In some embodiments, the patient is analyzed for responsiveness to cytokine therapy, where the selection of therapeutic agent is based on such analysis.

In various aspects and embodiments of the methods and compositions described herein, administering the therapeutic compositions can be effected or performed using any of the various methods and delivery systems known to those skilled in the art. The administering can be performed, for example, intravenously, orally, via implant, transmucosally, transdermally, intramuscularly, intrathecally, and subcutaneously. The delivery systems employ a number of routinely used pharmaceutical carriers. Usually the administration of the FXR agonist and optional statin is by oral administration.

In methods of use, an effective dose of a FXR agonist of the invention is administered alone or in a cocktail of agonists, or combined with additional active agents for the treatment of a condition as listed above. The effective dose may be from about 1 ng/kg weight, 10 ng/kg weight, 100 ng/kg weight, 1 μg/kg weight, 10 μg/kg weight, 25 μg/kg weight, 50 μg/kg weight, 100 μg/kg weight, 250 μg/kg weight, 500 μg/kg weight, 750 μg/kg weight, 1 mg/kg weight, 5 mg/kg weight, 10 mg/kg weight, 25 mg/kg weight, 50 mg/kg weight, 75 mg/kg weight, 100 mg/kg weight, 250 mg/kg weight, 500 mg/kg weight, 750 mg/kg weight, and the like. The dosage may be administered multiple times as needed, e.g. every 4 hours, every 6 hours, every 8 hours, every 12 hours, every 18 hours, daily, every 2 days, every 3 days, weekly, and the like. The dosage may be administered orally.

The compositions can be administered in a single dose, or in multiple doses, usually multiple doses over a period of time, e.g. daily, every-other day, weekly, semi-weekly, monthly etc. for a period of time sufficient to reduce severity of the inflammatory disease, which can comprise 1, 2, 3, 4, 6, 10, or more doses.

Determining a therapeutically or prophylactically effective amount of an agent according to the present methods can be done based on animal data using routine computational methods. The effective dose will depend at least in part on the route of administration.

### Pharmaceutical Compositions

The above-discussed compounds can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In some embodiments, the FXR agonist is formulated with a statin in a pharmaceutically acceptable excipient(s).

The subject formulations can be administered orally, subcutaneously, intramuscularly, parenterally, or other route, including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

Each of the active agents can be provided in a unit dose of from about 0.1 μg, 0.5 μg, 1 μg, 5 μg, 10 μg, 50 μg, 100 μg, 500 μg, 1 mg, 5 mg, 10 mg, 50, mg, 100 mg, 250 mg, 500 mg, 750 mg or more.

The FXR agonist may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

### Example 1

Obeticholic Acid, a Synthetic Bile Acid Agonist of the Farnesoid X Receptor, Attenuates Experimental Autoimmune Encephalomyelitis

Bile acids are ligands for the nuclear hormone receptor, farnesoid X receptor (FXR). The bile acid-FXR interaction regulates bile acid synthesis, transport and cholesterol metabolism. Recently, bile acid-FXR regulation has been reported to play an integral role in both hepatic and intestinal inflammation, and in atherosclerosis.

In this study, we found that FXR knockout mice had more disease severity in experimental autoimmune encephalomyelitis (EAE), an animal model of multiple sclerosis (MS). Obeticholic acid (6-ECDCA), a synthetic FXR agonist, is an orally available drug that is currently in clinical trials for the treatment of inflammatory diseases such as alcoholic hepatitis, nonalcoholic steatohepatitis, and primary biliary cirrhosis. When we treated mice exhibiting established EAE with 6-ECDCA, or the natural FXR ligand CDCA, clinical disease was ameliorated by 1) suppressing lymphocyte activation and proinflammatory cytokine production, 2) reducing CD4+ T cells and CD19+ B cells populations and their expression of negative checkpoint regulators PD1, PD-L1, and BTLA, 3) increasing CD8+ T cells and PD1, PDI-1, and BTLA expression, and 4) reducing VLA-4 expression in both the T and B cell populations. Moreover, adoptive transfer of 6-ECDCA or CDCA treated donor cells failed to transfer disease in naïve recipients. Thus, we show that FXR functions as a negative regulator in neuroinflammation and we highlight that FXR agonists represents a potential novel therapy for MS.

In this study, we explored the role of FXR in an animal model of multiple sclerosis (MS), experimental autoimmune encephalomyelitis (EAE). EAE is an inducible inflammatory disease of the central nervous system (CNS) mediated by myelin specific CD4+ T cells that is augmented by circulating myelin autoantibodies. Here we show that FXR knockout (FXRKO) mice exhibit enhanced EAE disease severity. Moreover, we found that the synthetic FXR agonist, 6-ECDCA, inhibits both active and passive EAE more effectively than the natural FXR ligand, CDCA in part by reducing IFN-gamma production and modulating both T and B trafficking and checkpoint inhibitors. Taken together, these findings demonstrate that FXR serves as a negative regulator of CNS autoimmune inflammation.

### Results:

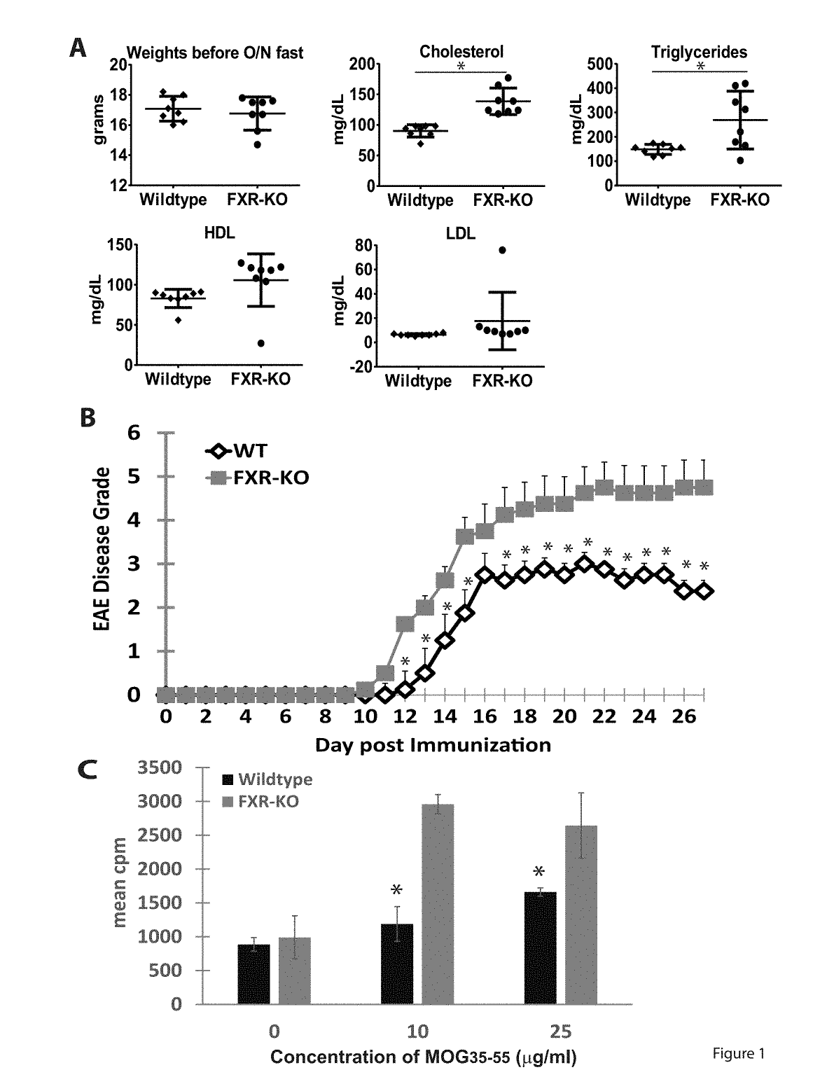

FXR Knockout mice have more severe EAE. Mice that are homozygous for the targeted Nr1h4 allele (FXR-KO) have been reported to display a proartherogenic serum lipoprotein profile that is characterized by elevated levels of serum cholesterol, triglycerides and bile acids. Before induction of EAE, we performed a lipid panel analysis of both wildtype and FXR-KO naïve mice that had been fasted overnight. As shown in FIG. 1A, we confirmed that the FXR-KO mice had statistically elevated levels of cholesterol and triglycerides and a trend towards increased high-density lipoprotein (HDL) levels compared to the wild-type mice. When both groups were immunized with MOG35-55, the FXR-KO mice had significantly more EAE disease severity than the wildtype mice (FIG. 1B). Proliferation assay of splenocytes indicated that splenocyte T cells from FXR-KO mice responded more robustly to MOG35-55 restimulation than from wildtype mice (FIG. 1D).

Oral administration of Obeticholic Acid is effective in treating EAE. Given that the lack of FXR expression worsened disease course of EAE, we wanted to determine if treatment of EAE with a FXR agonist would in turn be beneficial. We selected this synthetic orally administered FXR agonist as our treatment drug. We initially compared the route of administration of 6-ECDCA intraperitoneally versus orally to determine if one or both routes would be effective in treating established EAE. Additionally, to keep the study consistent, all treatment groups were dosed both orally and intraperitoneally with the vehicle alone or a combination of vehicle and 6-ECDCA.

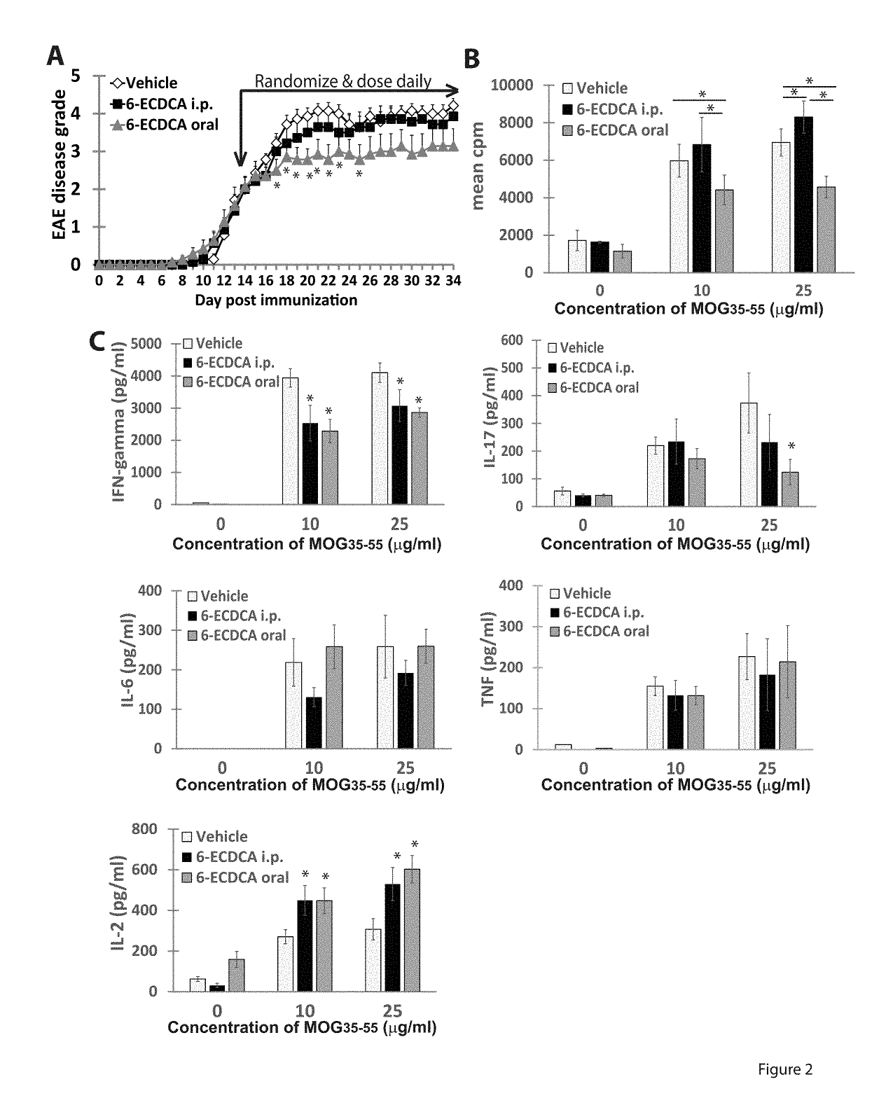

We found that following randomization of mice at the peak of disease, daily oral dosing of 6-ECDCA was most effective in attenuating established EAE (FIG. 2A). T cell proliferation to MOG35-55 was significantly reduced in mice treated orally with 6-ECDCA compared to mice treated with the vehicle control or treated intraperitoneally with 6-ECDCA (FIG. 2B). Splenocytes harvested from both orally and intraperitoneally 6-ECDCA treated mice had reduced IFN-gamma production in response to MOG35-55 compared to the vehicle control group, while oral treatment with 6-ECDCA reduced IL-17 production (FIG. 2C). IL-6 and TNF production were not significantly altered by 6-ECDCA, although production of IL-2 was enhanced.

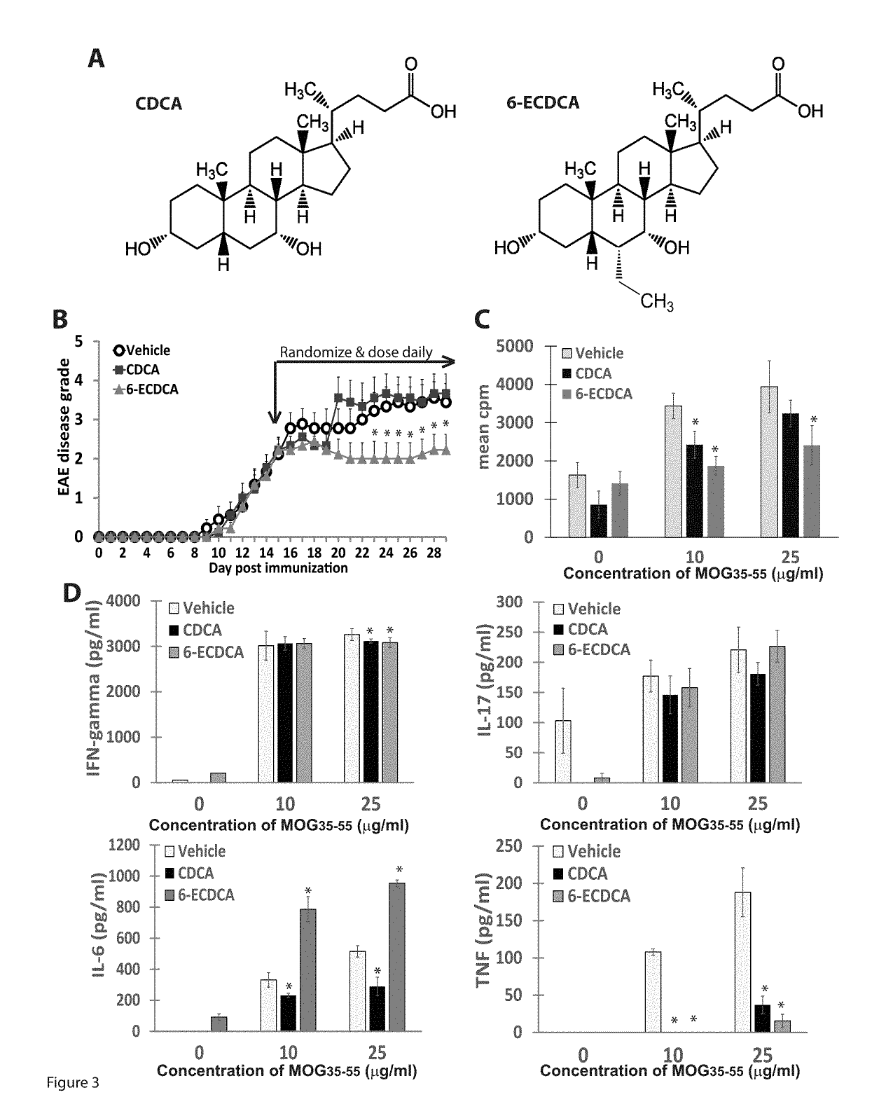

The synthetic FXR agonist, Obeticholic Acid, is more effective than the natural FXR ligand, Chenodeoxycholic Acid, in treating EAE. We next compared the efficacy of treating EAE with the natural bile acid FXR ligand, chenodeoxycholic acid (CDCA), versus 6-ECDCA, a CDCA derivative that contains an additional ethyl group (FIG. 3A). 6-ECDCA has ˜100-fold greater FXR agonistic activity than CDCA. Mice with established EAE were randomized and treated orally with daily doses of either vehicle, CDCA, or 6-ECDCA. As shown in FIG. 3B, daily oral treatment with 6-ECDCA was more effective than CDCA in ameliorating the average EAE disease grade in mice. T cell recall responses to MOG35-55 were significantly suppressed in both 6-ECDCA and CDCA treated mice (FIG. 3C). Both FXR agonists decreased IFN-gamma and TNF production (FIG. 3D). Interestingly, IL-6 production was decreased from CDCA treatment but increased with 6-ECDCA treatment, and IL-17 production was not significantly altered in this experiment.

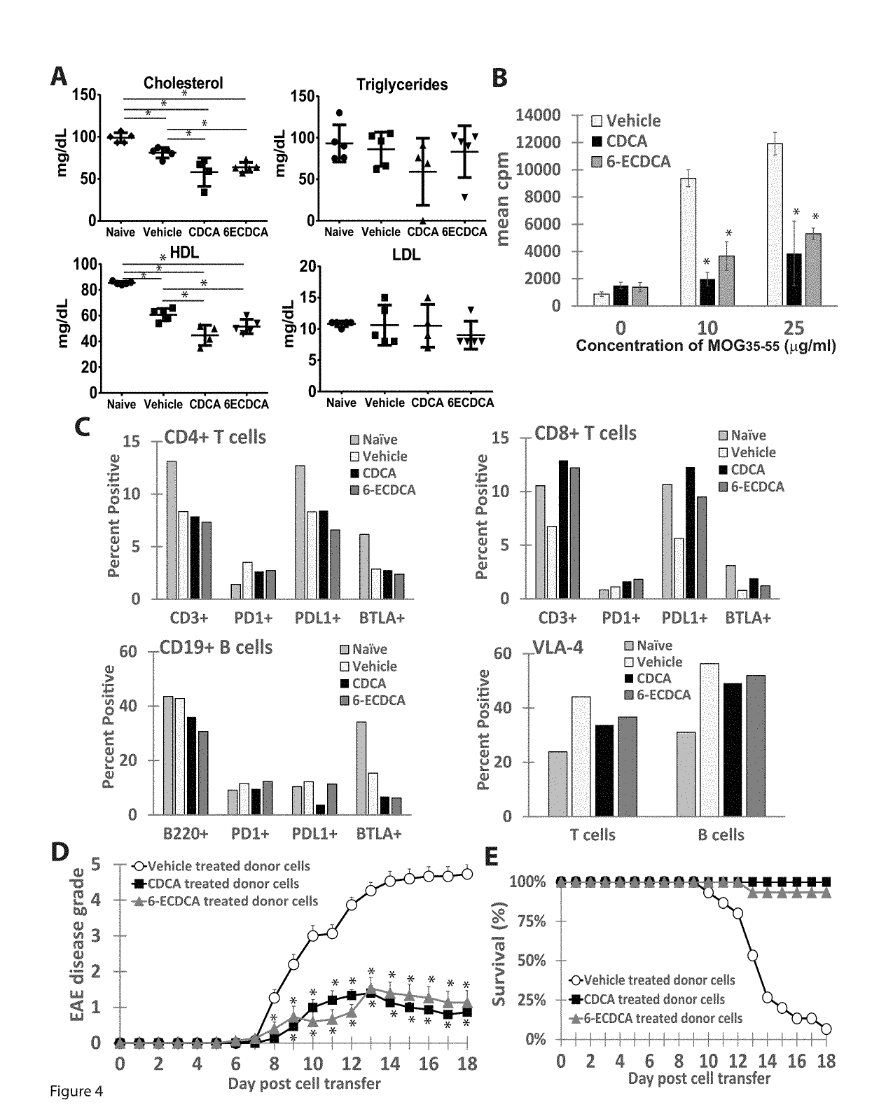

Obeticholic Acid and Chenodeoxycholic Acid alter the lymphocyte activation profile in EAE and prevent disease transfer. To investigate the underlying mechanism by which 6-ECDCA alters the immune cells in vivo during EAE, we examined several aspects following EAE immunization and eleven days of oral dosing with 6-ECDCA or CDCA compared to vehicle and naïve control mice. First, we assessed the serum lipid panel following an overnight fast. Both cholesterol levels and HDL levels were decreased in mice treated with 6-ECDCA and CDCA (FIG. 4A). The triglycerides and low-density lipoprotein (LDL) levels were not significantly altered. We next performed a proliferation assay with the combined splenocytes and lymphocytes harvested from each group. Both 6-ECDCA and CDCA treatment greatly reduced the recall responses of the MOG35-55-specific lymphocytes compared to the vehicle treatment (FIG. 4B).

FACScan analysis of the same splenocytes revealed a reduction in both CD3+CD4+ T cells (8.34% WT, 7.89% CDCA, 7.33% 6-ECDCA) and CD19+B220+ B cells (42.82% WT, 36.07% CDCA, 30.69% 6-ECDCA) while CD3+CD8+ T cells increased with treatment of 6-ECDCA and CDCA (6.75% WT, 12.92% CDCA, 12.22% 6-ECDCA) (FIG. 4C). Since the MOG35-55 proliferative responses in 6-ECDCA and CDCA treated cells were reduced, we hypothesized that the lymphocytes were potentially more prone to apoptosis. To address this possibility, we also stained for cell surface expression of three molecules involved in down-regulating the immune system by preventing the activation of lymphocytes and promoting apoptosis: PD1 (programmed cell death protein 1), PD-L1 (programmed death ligand 1), and BTLA (B and T lymphocyte attenuator). CD4+ T cells from spleens of 6-ECDCA treated mice had relatively reduced expression of PD1 (2.74%), PD-L1 (6.59%) and BTLA (2.38%) compared to CD4+ T cells from vehicle treated mice (3.5%, 8.32%, and 2.87%, respectively).

CD4+ T cells from spleens of CDCA treated mice had similarly reduced PD1 expression (2.64%) as 6-ECDCA treatment, but PD-L1 (8.43%) and BTLA (2.77%) expression were relatively unchanged. In contrast, with the increase in the CD8+ T cell population following treatment with 6-ECDCA and CDCA, the expression of PD1 (1.12% WT, 1.63% CDCA, 1.82% 6-ECDCA), PD-L1 (5.63% WT, 12.3% CDCA, 9.5% 6-ECDCA) and BTLA (0.78% WT, 1.92% CDCA, 1.21% 6-ECDCA) were all relatively increased compared to the CD8+ T cells from the vehicle control mice. As for the CD19+ B cells, CDCA treatment was more effective in reducing expression of PD1 (11.58% WT, 9.58% CDCA, 12.3% 6-ECDCA) and PD-L1 (12.21% WT, 3.79% CDCA, 11.34% 6-ECDCA). Both CDCA and 6-ECDCA appear to similarly decrease BTLA expression on CD19. B cells (6.76% CDCA, 6.23% 6-ECDCA) compared to vehicle treatment (12.21%).

We also stained for VLA-4 (CD29+CD49d+) expression on both T and B cells. The expression of VLA-4 (α4β1 integrin) on lymphocytes is necessary for the effective extravasation across the blood vessel endothelium into target organs such as the CNS. Tysabri, a monoclonal antibody directed against the α4 integrin subunit of VLA-4 is currently one of the most effective immunosuppressive drugs currently available for the treatment of MS. Here, we observed that VLA-4 expression was reduced in both T cells (44.12% WT, 33.78% CDCA, 36.67% 6-ECDCA) and B cells (56.35% WT, 49.16% CDCA, 51.98% 6-ECDCA) following treatment with 6-ECDCA and CDCA (FIG. 4C).

Together these results show that treatment with 6-ECDCA and CDCA can alter the activation and migration of both T and B cells. Finally, to determine if these treated cells can effectively transfer EAE disease, naïve recipients were injected with cells from donor mice that had been treated with either vehicle, CDCA, or 6-ECDCA for eleven days followed by an additional three days of MOG35-55 re-stimulation in the presence of rIL-12. FIG. 4D summarizes the EAE clinical scores, whereby mice receiving activated lymphocytes from 6-ECDCA or CDCA treated donor mice had significantly less severe disease than mice receiving activated lymphocytes from vehicle treated donor mice.

The objective of this study was to determine if the expression of the bile acid receptor, FXR, plays a critical role in the autoimmune demyelinating disease EAE. Here we found that loss of functional FXR exacerbated EAE disease in FXR-KO mice, whereas activation of FXR via a synthetic FXR-specific agonist, 6-ECDCA, effectively ameliorated both active and passive EAE. Administration of 6-ECDCA rendered MOG35-55 specific lymphocytes to be less reactive to MOG35-55 re-stimulation and suppressed several proinflammatory cytokines including IFN gamma, IL-17, and TNF. Physiologically, 6-ECDCA and CDCA reduced serum cholesterol and HDL levels in vivo. Immunologically, FXR agonists appear to decrease both activated CD4+ T cell and CD19+ B cell percentages but increase CD8+ T cells. The expression of PD1 is decreased in these CD4+ T cells, with 6-ECDCA having a more pronounced effect in decreasing PD-L1 expression on CD4+ T cells whereas CDCA was more effective in reducing PD-L1 expression on CD19. B cells. BTLA expression was also decreased on both CD4+ T cells and CD19+ T cells in mice treated with the FXR agonists. In contrast, FXR agonists appear to increase the percentages of MOG35-55 activated CD8+ T cell. Moreover, PD-1, PD-L1, and BTLA expression were all upregulated in these activated CD8+ T cells. VLA-4 expression, the cell surface molecule critical for lymphocyte extravasation from blood vessels into target organs such as the CNS, was decreased in both T and B cells following treatment with the FXR agonists. Finally, adoptive transfer of MOG35-55-specific lymphocytes treated in vivo with the FXR agonists failed to induce EAE in naïve recipients.

The costimulatory pathway of PD-1 and PD-L1/PD-L2 delivers inhibitory signals that regulate the balance among T cell activation, tolerance, and immune-mediated tissue damage during interactions with self-antigens, chronic viral infections, and tumors. PD-1 expression is normally up-regulated on T cells upon activation, whereas the effectors of PD-1 ligation on T cells is evident as early as two hours after activation. Binding of PD-1 with either of its ligands during TCR signaling can block T-cell proliferation, cytokine production and cytolytic function, and impair T cell survival.

BTLA is another negative checkpoint regulator with expression limited to B cells, T cells, mature dendritic cells, and macrophages (32). BTLA negatively regulates T cell activation, and BTLA engagement leads to peripheral tolerance and pro-survival function. Our observation that both CD4+ T cells and CD19+ B cells had decreased expression of PD-1, PD-L1 and BTLA following treatment with 6-ECDCA and CDCA indicates that modulation of these two MOG35-55 activated cell populations leads to decreased T cell proliferation and that these populations may be more prone to apoptosis.

MS is characterized by an inflammatory infiltrate that includes CD4+ and CD8+ T cells. Clonal expansion of CD8+ T cells appear to persist within active lesions and in the cerebrospinal fluid and blood in MS. Myelin-specific CD8+ T cells have been shown to play a pathogenic role in EAE. However, more recently, myelin-specific autoregulatory CD8+ T cells have been shown to inhibit EAE, through the suppression of CD4+ T cell and/or antigen-presenting cell function by direct killing in an antigen-specific manner. The fact that both FXR agonists increased the CD8+ T cell population in vivo concurrently with increased PD1, PD-L1 and BTLA expression, suggests that a subset of autoregulatory CD8+ T cells may be enhanced.

Arresting lymphocyte trafficking has been the approach for two currently approved MS disease modifying therapies. Tysabri (Natalizumab), a monoclonal antibody directed against α4 integrin, prevents the extravastion of circulating activated lymphocytes across the blood-brain barrier into the CNS. In contrast, the first orally available MS therapeutic Gilenya (Fingolimod), a sphingosine 1-phosphate receptor agonist, sequesters activated lymphocytes within the secondary lymphoid organs resulting in severe lymphopenia. Here we show that treating MOG35-55-immunized mice with an FXR agonist also reduces the expression of VLA-4 expression on both T and B cells implicating a role in modulating cell trafficking during lymphocyte activation.

Regarding orally available treatments of MS the finding here that intraperitioneal injection was less effective than oral administration was unexpected. The oral activity of obeticholic acid suggests further modulation of the drug by gut flora or gut enzymes, or the importance of hepatic uptake. Inflammation impairs reverse cholesterol transport by cholesterol efflux from peripheral cells, such as lipid-laden foam cells, onto circulating HDL for transport to the liver for secretion into bile and feces.

Impairment of reverse cholesterol transport may contribute to atherosclerosis in chronic inflammatory diseases including obesity, metabolic syndrome, and type 2 diabetes. The bile acid biosynthesis pathway plays an important role in maintaining cholesterol homeostasis in mammals. The conversion of cholesterol to bile acids accounts for the catabolism of about 50% cholesterol in the body, and bile acids are also required for the disposal of 40% of cholesterol in feces. Bile acids are reabsorbed in the ileum by active transport systems and are transported back to the liver through the portal venous circulation. The enterohepatic circulation of bile acids is extremely efficient, with only 5% bile acids lost in feces which is compensated for by biosynthesis from cholesterol. This feedback mechanism not only regulates bile acid synthesis and bile flow, but is also important in regulating cholesterol synthesis by inhibiting the rate-limiting enzyme, 3-hydroxy-3-methylglutaryl-CoA (HMG-CoA) reductase and hepatic uptake of LDL cholesterol by LDL receptor.

We have other have previously reported on the benefits of using cholesterol lowering HMG-CoA reductase inhibitors, statins, for the treatment of EAE. Blockade of HMGCoA reductase inhibits proinflammatory T helper cell response during EAE by regulating isoprenoid availability through the mevalonate pathway). Targeting the bile acid synthesis pathway may be another approach in blocking HMG-CoA reductase to further inhibit the proinflammatory T cell response in EAE and MS. FXR is not the first nuclear hormone receptor involved in regulating cholesterol turnover and targeted for controlling proinflammatory responses in EAE. Peroxisome proliferator activated receptors (PPAR), another member of the nuclear hormone receptor subfamily, also regulates whole body lipid and glucose homeostasis and controls inflammatory responses. We have reported that PPAR alpha and PPAR delta serve as important molecular brakes on T cell immunity for the control of CNS inflammation. Furthermore several PPAR agonists directed towards PPAR alpha, PPAR beta/delta, and PPAR gamma have all shown efficacy in treating EAE.

### Materials and Methods

Mice and EAE Induction. C57BL/6J and FXRKO (B6.129x1(FVB)-Nr1h4tm1Gonz/J, #0007214) female mice were purchased from the Jackson Laboratory. Animal experiments were approved by, and performed in compliance with, the National Institute of Health guidelines of the Institutional Animal Care and Use Committee at Stanford University. For induction of active and adoptive transfer of EAE, 8- to 12-week-old female mice were immunized subcutaneously with 100 μg of MOG35-55 emulsified in complete Freund's adjuvant (CFA), consisting of incomplete Freund's Adjuvant and 0.4 mg of heat inactivated Mycobacterium tuberculosis, strain H37 RA (Difco Laboratories). On days 0 and 2, mice were also injected intravenously or intraperitoneally with 400 ng pertussis toxin in 0.2 ml 1×PBS. Adoptive transfer requires harvesting lymph nodes and spleens ten days after immunization. Combined lymphocytes and splenocytes are cultured in bulk, at 5×10⁷ cells/ml for seventy-two hours in the presence of MOG35-55 (20 μg/ml) and rIL-12 (10 ng/ml, R&D systems). Cells are harvested and washed twice before transfer of 3×10⁷ cells in 0.2 ml 1×PBS into naive 6-7 week old female mice, intraperitoneally. Clinical disease was monitored daily using the following scoring system: 0, no disease; 1, limp tail; 2, hind limb weakness; 3, hind limb paralysis; 4, hind limb and forelimb paralysis; 5, death.

Reagents MOG p35-55 (MEVGWYRSPFSRVVHLYRNGK) was obtained from Genemed Synthesis (San Antonio, Tex.). 6α-Ethyl-chenodeoxycholic acid (6-ECDCA, Obeticholic acid) was initially obtained from Caymen Chemicals and later from AdipoGen International. Chenodeoxycholic acid (CDCA) was obtained from Sigma. Both were solubilized in 100% dimethyl sulfoxide (DMSO) at a stock concentration of 25 mg/ml and further diluted with Phosphate Buffered Saline (1×). Mice were given 200 μl of 6-ECDCA (5 mg/kg) or CDCA (5 mg/kg) daily, either intraperitoneally or orally where indicated. Vehicle control contained equivalent amounts of DMSO and 1×PBS.

Proliferation and cytokine assays. Splenocytes and lymph nodes were harvested and stimulated with increasing concentrations of MOG35-55 for a total of 72 hours. For assessment of proliferation, 1 μCi of ³H-thymidine was added to each well for the final 18-24 hours of culture, and incorporation of radioactivity was measured by using a Betaplate scintillation counter. Cytokine assays were performed on culture supernatants after 72 hours of culture by using the IL-2, IL-6, IFNgamma, and TNF BD OptEIA™ Mouse ELISA kits (BD Biosciences) or Mouse IL-17 DuoSet ELISA Development kit (R&D Systems).

Flow cytometry Cells were stained according to standard protocols, run on a FACScan flow cytometer (BD Biosciences), and analysed with CellQuest software (BD Immunocytometry Systems). The antibody conjugates used were PerCPCy5.5 anti-CD3, FITC anti-CD4, PE anti-CD8, PE anti-B220, PerCp-Cy5.5 anti-B220, FITC anti-CD19, PE anti-PD1, PE anti-PD-L1, PE anti-BTLA, PE anti-CD29, and FITC anti-CD49d (BD Pharmingen & eBioscience)

Serum Lipid Panel Analysis. Serum from mice fasted overnight was submitted to the Diagnostic Laboratory at Stanford University, Department of Comparative Medicine for a full lipid panel analysis.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

What is claimed is:

1. A method for treating an inflammatory demyelinating disease in a patient, the method comprising:
administering to said patient a therapeutically effective dose of one or more agonist(s) of farnesoid X receptor (FXR).

2. The method of claim 1, wherein the FXR agonist is a synthetic agonist.

3. The method of claim 2, wherein the agonist is obeticholic acid (6-ECDCA).

4. The method of claim 1, wherein the FXR agonist is a natural agonist.

5. The method of claim 4, wherein the agonist is chenodeoxycholic acid (CDCA).

6. The method of any one of claim 1, wherein the inflammatory demyelinating disease is multiple sclerosis.

7. The method of claim 1, wherein the administering step is oral administration.

8. The method of claim 1, further comprising administering an effective dose of a statin.

9. The method of claim 1, further comprising administering an additional therapeutic agent for treatment of an inflammatory demyelinating disease.

10. A composition comprising a package comprising an FXR agonist and a package insert or label that indicates that the FXR agonist is to be administered to a patient for the treatment of a demyelinating autoimmune disease.

11. The method of claim 1, wherein the patient is patient is analyzed for responsiveness to cytokine therapy, and where the selection of therapeutic agent is based on such analysis.
